(19) United States
(12) Patent Application Publication
(10) Pub. No.: US 2024/0366565 A1
(43) Pub. Date: Nov. 7, 2024

(54) Use of Verteporfin to Modulate Wound Healing After an Ocular Surgical Procedure or Ocular Injury

(71) Applicant: The Board of Trustees of the Leland Stanford Junior University, Stanford, CA (US)

(72) Inventors: Wendy Liu, Palo Alto, CA (US); David Myung, Saratoga, CA (US)

(21) Appl. No.: 18/690,492

(22) PCT Filed: Sep. 9, 2022
(86) PCT No.: PCT/US2022/043063

§ 371 Date: Mar. 8, 2024

Related U.S. Application Data
(60) Provisional application No. 63/242,192, filed on Sep. 9, 2021.

(51) Int. Cl. A61K 31/409; A61K 9/00; A61P 27/02

(52) U.S. Cl. CPC A61K 31/409; A61K 9/0048; A61P 27/02

(57) ABSTRACT

Methods for reducing the risk of fibrosis or scarring after an ocular surgical procedure or ocular injury are disclosed. In particular, one or more therapeutically effective doses of verteporfin are administered to a subject before, during, or after an ocular surgical procedure or after an ocular injury to treat or reduce the risk of developing fibrosis or scarring.

### BACKGROUND

The success of ocular surgical procedures, such as glaucoma filtration surgery, depends on wound healing. For example, the major limitation of the long-term success of glaucoma filtration surgery is scarring and abnormal wound healing of the subconjunctival space. Currently, the most commonly used agents, 5-fluorouracil and mitomycin C (MMC), are antimetabolites, which can cause cytotoxic side effects. Finding effective anti-fibrotic agents with minimal side effects has been the holy grail to enhancing surgical success. In addition, scarring after ocular injury can have devastating consequences. For example, burns and injury to the cornea can lead to permanent scarring of the cornea and loss of vision. In the case of burns and severe injuries, preserving vision requires preventing fibrosis during the healing process, and facilitating transparent, “scarless” tissue regeneration.

Thus, there is an ongoing and unmet need for an improved approach to modulate wound healing after ocular surgical procedures and ocular injury, including after glaucoma surgery and corneal injury.

### SUMMARY

Methods for reducing the risk of fibrosis or scarring after an ocular surgical procedure or ocular injury are disclosed. In particular, one or more therapeutically effective doses of verteporfin are administered to a subject before, during, or after an ocular surgical procedure or after an ocular injury to treat or reduce the risk of developing fibrosis in ocular tissues.

In one aspect, a method of treating fibrosis or reducing risk of developing fibrosis or scarring in an eye of a subject after an ocular injury or ocular surgery is provided, the method comprising administering a therapeutically effective amount of verteporfin to the subject.

In certain embodiments, the ocular surgery is glaucoma surgery.

In certain embodiments, the ocular injury is caused by physical trauma or a chemical exposure.

In certain embodiments, the ocular injury or ocular surgery causes a corneal wound or a conjunctival wound.

In certain embodiments, the ocular injury increases risk of corneal or conjunctival fibrosis or scarring.

In certain embodiments, multiple cycles of treatment are administered to the subject.

In certain embodiments, the verteporfin is administered to the subject intermittently.

In certain embodiments, the verteporfin is administered no more frequently than biweekly.

In certain embodiments, the verteporfin is administered for at least one month, at least two months, or at least three months.

In certain embodiments, the verteporfin is administered in one or more doses (e.g., topically or by subconjunctival injection). In some embodiments, each administration provides a dose ranging from 0.001 mg to 10 mg of verteporfin, including any dose within this range such as 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 1 mg, 2 mg, 3 mg, 4 mg, 5 mg, 6 mg, 7 mg, 8 mg, 9 mg, or 10 mg. In some embodiments, each administration provides a dose ranging from 0.01 mg to 1 mg of verteporfin. In some embodiments, the verteporfin is administered in a volume ranging from 0.1 ml to 1 ml.

In certain embodiments, the verteporfin is administered locally under the conjunctiva or topically on the cornea.

In certain embodiments, the verteporfin is administered before, during, or after an ocular surgical procedure that increases the risk of developing fibrosis in ocular tissues, or after ocular injury that increases the risk of scarring in ocular tissues.

In certain embodiments, the ocular surgical procedure comprises incision of the conjunctiva.

In certain embodiments, the ocular surgical procedure is glaucoma filtration surgery (trabeculectomy), minimally invasive glaucoma surgery (MIGS), insertion of a glaucoma drainage implant, insertion of a Xen gel stent, or insertion of other stents or devices in the subconjunctival space, a revision of a trabeculectomy, a revision of MIGS, a revision of a glaucoma drainage implant, a revision of a Xen gel stent, or revision of other stents or devices in the subconjunctival space. In some embodiments, the ocular surgical procedure comprises incisional surgery or bleb needling to revise a previous trabeculectomy, previous tube shunt surgery, or other previous incisional surgery.

In certain embodiments, verteporfin is administered before, during, or after an ocular surgical procedure or ocular injury; and subsequent administrations of verteporfin are no more frequent than biweekly up to postoperative month three. In some embodiments, verteporfin is administered up to five times subsequently. In some embodiments, the method further comprises administering verteporfin after postoperative month three. In some embodiments, the method further comprises administering verteporfin one, two, three, four, five, six, seven, eight, or nine additional times after the fifth administration of verteporfin. In some embodiments, the additional doses of verteporfin are administered monthly.

In certain embodiments, the administration of the verteporfin is performed in conjunction with bleb needling or other manipulation of the conjunctiva, performed in the office or in the operating room.

In certain embodiments, the verteporfin is administered after an ocular injury that increases the risk of developing fibrosis in ocular tissues such as the cornea.

In another aspect, a method of treating fibrosis in ocular tissues is provided, the method comprising: identifying a subject who has fibrosis in ocular tissues or is at risk of developing fibrosis or scarring in ocular tissues after a previous ocular surgery or ocular injury; administering to the subject a first dose of verteporfin; and administering to the subject subsequent doses of verteporfin no more frequently than biweekly, wherein up to five doses of verteporfin are administered to the subject.

In certain embodiments, the verteporfin is administered in one or more doses (e.g., topically or by subconjunctival injection). In some embodiments, a dose is 0.001 mg to 10 mg of verteporfin, including any dose within this range such as 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 1 mg, 2 mg, 3 mg, 4 mg, 5 mg, 6 mg, 7 mg, 8 mg, 9 mg, or 10 mg. In some embodiments, a dose ranges from 0.01 mg to 1 mg of verteporfin. In some embodiments, the verteporfin is administered in a volume ranging from 0.1 ml to 1 ml.

In certain embodiments, the verteporfin is administered topically on the cornea or under the conjunctiva.

In certain embodiments, the ocular surgical procedure is glaucoma filtration surgery (trabeculectomy), minimally invasive glaucoma surgery (MIGS), insertion of a glaucoma drainage implant, insertion of a Xen gel stent, or insertion of other stents or devices in the subconjunctival space, a revision of a trabeculectomy, a revision of MIGS, a revision of a glaucoma drainage implant, a revision of a Xen gel stent, or revision of other stents or devices in the subconjunctival space, or other conditions where the conjunctiva is incised. In some embodiments, the ocular surgical procedure comprises incisional surgery or bleb needling to revise a previous trabeculectomy, previous tube shunt surgery, or other previous incisional surgery.

In certain embodiments, the method further comprises administering one, two, three, four, five, six, seven, eight, or nine additional doses of verteporfin after the fifth dose of verteporfin. In some embodiments, the additional doses are administered monthly.

In certain embodiments, each administration of verteporfin is performed in conjunction with bleb needling or other manipulation of the conjunctiva, performed in the office or in the operating room.

In certain embodiments, a method of reducing corneal scarring in an eye of a subject after a corneal injury is provided, the method comprising administering a therapeutically effective amount of verteporfin to the subject.

In another aspect, a composition comprising verteporfin for use in a method of treating fibrosis or scarring in ocular tissues or reducing risk of developing fibrosis or scarring in ocular tissues is provided. In some embodiments, the fibrosis is conjunctival fibrosis.

In another aspect, a composition comprising verteporfin for use in a method of treating a subject after a corneal injury to reduce corneal scarring is provided.

In another aspect, a composition comprising verteporfin for use in a method of treating fibrosis or scarring in ocular tissues is provided, the method comprising: identifying a subject who has fibrosis in ocular tissues or is at risk of developing fibrosis or scarring in ocular tissues after a previous ocular surgery or ocular injury; administering to the subject a first dose of verteporfin; and administering to the subject subsequent doses of verteporfin no more frequently than biweekly, wherein up to five doses of verteporfin are administered to the subject.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Verteporfin reduces expression of α-smooth muscle actin in hMSCs. Human mesenchymal stem cells (hMSCs) were plated in culture. The next day, the following treatments were added in duplicate: 1 μg/mL Verteporfin, 10 μg/mL Verteporfin, 10 ng/ml transforming growth factor beta-1 (TGFB-1), 1 μg/mL Verteporfin+10 ng/mL TGFB-1, 10 μg/mL Verteporfin+10 ng/ml TGFB-1, Dimethyl sulfoxide (DMSO), and cells without treatment. After 24 hours, the medium was removed, the wells were washed with PBS and then the RNA of the cells were extracted using Trizol and a Quiagen kit. Quantitative polymerase chain reaction (qPCR) was performed. TGFB-1 activation leads to the differentiation of mesenchymal stem cells into myofibroblasts, inducing expression of α-smooth muscle actin (α-SMA) and the extracellular matrix proteins such as collagen which may lead to scarring. The addition of TGFB-1 statistically increased the expression of alpha-SMA (ACTA-2) after 24 hours. Treating cells with TGFB-1 and Verteporfin statistically reduced the expression of alpha-SMA in a dose dependent manner, suggesting that verteporfin may reduce scarring and fibrosis.

FIG. 2. Results of immunostaining alpha smooth muscle actin in hMSCs. The hMSCs, were treated with 10 ng/ml TGFB-1 with or without verteporfin and DMSO for 24 hours. The next day, the cells were fixed and stained for alpha smooth muscle actin (ASMA). Results show that Verteporfin reduces the expression of ASMA, suggesting that it can reduce scarring and the fibrotic response.

FIG. 3. Verteporfin does not increase expression of ki67 in hMSCs. The hMSCs were cultured on plates. The next day, the treatments were added in duplicate: Verteporfin 1 μg/mL, Verteporfin 10 μg/mL, DMSO, and cells without treatment. After 24 hours, the medium was removed, the wells were washed with PBS and then the RNA of the cells were extracted using Trizol and Quiagen kit. qPCR was performed. The addition of Verteporfin did not increase the expression of ki67 (Mki67), a marker of cell proliferation.

FIG. 4. Verteporfin is not cytotoxic to hMSCs. The hMSCs were treated with verteporfin at concentrations of 1 μg/mL and 10 μg/mL for 24 hours. Cells were treated with 10% DMSO for 40 minutes. The next day, the live/dead solution was added to the cells. As shown in FIG. 4, verteporfin at 1 μg/mL and 10 μg/mL is not cytotoxic to hMSCs.

FIGS. 5A-5B. Efficacy of verteporfin in reducing scarring and increasing bleb survival. The efficacy of verteporfin in reducing scarring and increasing bleb survival was evaluated in a rabbit model of glaucoma filtration surgery. Glaucoma filtration surgery was performed in both eyes of New Zealand White rabbits. In one eye, various concentrations of verteporfin or Mitomycin C was injected subconjunctivally into the bleb after surgery. In the contralateral eye, BSS was injected after surgery as a control. Bleb appearance and survival were evaluated postoperatively at least twice a week. Bleb survival was the primary endpoint. The results show a trend towards increased survival in the verteporfin treated groups over the control (FIG. 5A). There were no post-surgical complications in all groups, including corneal edema, bleb leak, anterior chamber inflammation, iris involvement, cataract or infection (FIG. 5B).

FIG. 6. Efficacy of verteporfin in reducing corneal scarring. Anterior lamellar keratectomy (ALK) with a cut depth of around 50% was performed in a rat model of corneal injury. 10 μg/mL verteporfin (in Provisc gel) was subconjunctivally injected after ALK on Day 0. Eyes treated with verteporfin showed reduced corneal scarring at day 3 compared with eyes that were not treated.

### DETAILED DESCRIPTION OF EMBODIMENTS

Methods for reducing the risk of fibrosis and/or scarring after an ocular surgical procedure or ocular injury are disclosed. In particular, one or more therapeutically effective doses of verteporfin are administered to a subject before, during, or after an ocular surgical procedure or after an ocular injury to treat or reduce the risk of developing fibrosis or scarring in ocular tissues.

Before the present methods of using verteporfin for reducing the risk of fibrosis or scarring after an ocular surgical procedure or ocular injury are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a drug” includes a plurality of such drugs and reference to “the drug” includes reference to one or more drugs and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with fibrosis in ocular tissues) as well as those in which prevention is desired (e.g., those with increased susceptibility to developing fibrosis or scarring in ocular tissues because of having an ocular surgical procedure or ocular injury).

By “therapeutically effective dose or amount” of verteporfin is intended an amount that, when administered, as described herein, brings about a positive therapeutic response, such as improved recovery from an ocular surgical procedure or ocular injury, including reducing or preventing fibrosis and/or scarring. In particular, a “therapeutically effective dose or amount” of verteporfin may reduce or prevent fibrosis in ocular tissues after an ocular surgical procedure or ocular injury. Additionally, treatment may reduce eye pain, redness, and/or scarring. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Substantially purified” generally refers to isolation of a component such as a substance (e.g., compound, drug) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

The terms “recipient”, “individual”, “subject”, “host”, and “patient”, are used interchangeably herein and refer to any vertebrate subject for whom diagnosis, treatment, or therapy is desired, particularly humans. By “vertebrate subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

### Pharmaceutical Compositions

Verteporfin can be formulated into pharmaceutical compositions, optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the verteporfin, or other components of the preparation. Suitable antioxidants for use include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the verteporfin (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000. Examples of such excipients may include but are not limited to hyaluronic acid, collagen, carboxymethylcellulose, alginate, hypromellose, polyvinyl alcohol, polyethylene glycol, chondroitin sulfate, as well as combinations and/or derivatives thereof.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional compositions include those for oral, topical, ocular, or localized delivery. Formulations suitable for oral, topical, ocular, or localized administration may be prepared through use of appropriate suspending agents, solubilizers, thickening agents, stabilizers, and preservatives. Such formulations may be utilized as liquid drops or with a means to provide continuous administration, for example, incorporation into slow-release pellets or controlled-release patches.

The pharmaceutical preparations herein can also be housed in a syringe, an implantation device, or the like, depending upon the intended mode of delivery and use. Preferably, the compositions comprising the verteporfin are in unit dosage form, meaning an amount of a composition appropriate for a single dose, in a premeasured or pre-packaged form.

The compositions herein may optionally include one or more additional agents, such as analgesics, anti-inflammatory agents, anti-thrombotic/anti-fibrotic agents, antimetabolites, antibodies, and/or other medications used to treat a subject for fibrosis in ocular tissues. Compounded preparations may include the verteporfin and one or more other agents, such as, but not limited to, analgesics, including acetaminophen, non-steroidal anti-inflammatory agents (NSAIDs) such as aspirin, ibuprofen and naproxen, COX-2 inhibitors such as rofecoxib, celecoxib, and etoricoxib, and opioids such as morphine, codeine, oxycodone, hydrocodone, dihydromorphine, and pethidine; anti-inflammatory agents, including steroids such as prednisolone, betamethasone, dexamethasone, fluorometholone, hydrocortisone, and medrysone; anti-thrombotic/anti-fibrotic agents, including tissue plasminogen activator, urokinase, pirfenidone, and saratin; antimetabolites, including mitomycin C and 5-fluorouracil; and antibodies, including anti-VEGF monoclonal antibodies such as bevacizumab, and anti-TGF-β2 monoclonal antibodies such as lerdelimumab; and the like. Alternatively, such agents can be contained in a separate composition from the composition comprising the verteporfin and co-administered concurrently, before, or after the composition comprising the verteporfin.

### Administration

At least one therapeutically effective dose of verteporfin is administered. By “therapeutically effective dose or amount” of verteporfin is intended an amount that, when administered, as described herein, brings about a positive therapeutic response, such as improved recovery from an ocular surgical procedure or ocular injury, including reducing or preventing fibrosis. In particular, a “therapeutically effective dose or amount” of verteporfin may reduce or prevent fibrosis in ocular tissues after an ocular surgical procedure or ocular injury. Additionally, treatment may reduce eye pain, redness, and/or scarring.

The subject methods can be used to treat fibrosis and/or reduce or prevent fibrosis and/or scarring in ocular tissue including, without limitation, surface ocular tissue such as the cornea, conjunctiva, and sclera; and deeper ocular tissue such as tissue of the uvea, including the choroid, ciliary body, pigmented epithelium, and iris; and retina tissue. In some embodiments, verteporfin is used to treat a corneal wound or a conjunctival wound resulting from an ocular surgical procedure or ocular injury.

In some embodiments, one or more therapeutically effective doses of verteporfin are administered to a subject before, during, or after an ocular surgical procedure or injury to prevent or reduce the risk of developing fibrosis. The ocular surgical procedure may comprise incision of the conjunctiva. In some embodiments, the ocular surgical procedure is glaucoma filtration surgery (trabeculectomy), minimally invasive glaucoma surgery (MIGS), insertion of a glaucoma drainage implant, insertion of a Xen gel stent, or insertion of other stents or devices in the subconjunctival space, a revision of a trabeculectomy, a revision of MIGS, a revision of a glaucoma drainage implant, a revision of a Xen gel stent, or revision of other stents or devices in the subconjunctival space. In some embodiments, the surgical procedure is incisional surgery or bleb needling to revise a previous trabeculectomy, previous glaucoma drainage implant surgery, previous minimally invasive glaucoma surgery, or other previous incisional surgery.

In some embodiments, one or more therapeutically effective doses of verteporfin are administered to a subject after an ocular injury to prevent or reduce the risk of developing fibrosis or scarring. Verteporfin can be used to treat any type of eye injury that increases the risk of developing fibrosis or scarring in ocular tissue, including eye injuries resulting from physical trauma or a chemical exposure. In some embodiments, at least one therapeutically effective dose of verteporfin is administered to reduce corneal scarring in an eye of a subject after a corneal injury.

In certain embodiments, multiple therapeutically effective doses of verteporfin will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, the verteporfin will be administered twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of verteporfin are administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. In certain embodiments, the verteporfin is administered no more frequently than biweekly. In certain embodiments, the verteporfin is administered for at least one month, at least two months, or at least three months. The verteporfin can be administered by any acceptable route of administration as noted herein below.

In other embodiments, the pharmaceutical composition comprising the verteporfin is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

Verteporfin (again, preferably provided as part of a pharmaceutical preparation) can be administered alone or in combination with one or more other therapeutic agents, such as analgesics, anti-inflammatory agents, anti-thrombotic/anti-fibrotic agents, antimetabolites, antibodies, and/or other medications used to treat a subject for fibrosis, including, but not limited to, analgesics, including acetaminophen, non-steroidal anti-inflammatory agents (NSAIDs) such as aspirin, ibuprofen and naproxen, COX-2 inhibitors such as rofecoxib, celecoxib, and etoricoxib, and opioids such as morphine, codeine, oxycodone, hydrocodone, dihydromorphine, and pethidine; anti-inflammatory agents, including steroids such as prednisolone, betamethasone, dexamethasone, fluorometholone, hydrocortisone, and medrysone; anti-thrombotic/anti-fibrotic agents, including tissue plasminogen activator, urokinase, pirfenidone, and saratin; antimetabolites, including mitomycin C and 5-fluorouracil; and antibodies, including anti-VEGF monoclonal antibodies such as bevacizumab, and anti-TGF-2 monoclonal antibodies such as lerdelimumab; and the like, and other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

The verteporfin can be administered prior to, concurrent with, or subsequent to other agents. If provided at the same time as other agents, verteporfin can be provided in the same or in a different composition. Thus, verteporfin and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising verteporfin and a dose of a pharmaceutical composition comprising at least one other agent, such as another drug for treating ocular fibrosis, which in combination comprise a therapeutically effective dose, according to a particular dosing regimen. Similarly, verteporfin and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Toxicity can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

The pharmaceutical compositions comprising the verteporfin and optionally other agents may be administered using the same or different routes of administration in accordance with any medically acceptable method known in the art. Suitable routes of administration include parenteral administration, such as intraocular, subcutaneous (SC), intraperitoneal (IP), intramuscular (IM), intravenous (IV), or infusion, and oral, pulmonary, topical, and transdermal. In some embodiments, the pharmaceutical composition comprising the verteporfin is administered topically or by injection locally into the eye. In some embodiments, the verteporfin is administered locally under the conjunctiva. In some embodiments, the verteporfin is administered by subconjunctival injection. In some embodiments, the verteporfin is administered topically as eye drops, on a patch, or in a gel. For example, the verteporfin may be administered topically on the cornea.

In certain embodiments, the verteporfin is administered in one or more doses (e.g., topically on the cornea or by subconjunctival injection). In some embodiments, a dose is 0.001 mg to 10 mg of verteporfin, including any dose within this range such as 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 1 mg, 2 mg, 3 mg, 4 mg, 5 mg, 6 mg, 7 mg, 8 mg, 9 mg, or 10 mg. In some embodiments, a dose ranges from 0.01 mg to 1 mg of verteporfin. In some embodiments, the verteporfin is administered in a volume ranging from 0.1 ml to 1 ml.

In certain embodiments, a first dose of verteporfin is administered before, during, or after an ocular surgical procedure; and subsequent doses of verteporfin are administered no more frequently than biweekly up to postoperative month three. In some embodiments, up to five subsequent doses of verteporfin are administered. In some embodiments, the method further comprises administering additional doses of verteporfin after postoperative month three. In some embodiments, the method further comprises administering one, two, three, four, five, six, seven, eight, or nine additional doses of verteporfin after the fifth dose of verteporfin. In some embodiments, the additional doses of verteporfin are administered monthly. In certain embodiments, the administration of the verteporfin is performed in conjunction with bleb needling or other manipulation of the conjunctiva, which may be performed in an office or in an operating room.

Factors influencing the respective amount of the various compositions to be administered include, but are not limited to, the mode of administration, the frequency of administration (i.e., daily, or intermittent administration, such as twice- or thrice-weekly), the type of ocular surgery, the particular location of ocular injury, the severity of the ocular injury, the degree of risk of ocular fibrosis or scarring, the history of ocular disease, whether the individual is undergoing concurrent therapy with another therapeutic agent, and the age, height, weight, health, and physical condition of the individual undergoing therapy. Generally, a higher dosage of an agent is preferred with increasing weight of the subject undergoing therapy.

### Kits

Kits may comprise one or more containers of the pharmaceutical compositions, described herein, comprising verteporfin. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with a pharmaceutical composition.

In addition to the above components, the subject kits may further include (in certain embodiments) instructions for practicing the subject methods (i.e., instructions for treating or reducing risk of developing fibrosis and/or scarring in ocular tissue with verteporfin, as described herein). These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-39 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A method of treating fibrosis or reducing risk of developing fibrosis or scarring in an eye of a subject after an ocular injury or ocular surgery, the method comprising administering a therapeutically effective amount of verteporfin to the subject.

2. The method of aspect 1, wherein the ocular surgery is glaucoma surgery.

3. The method of aspect 1, wherein the ocular injury is caused by physical trauma or a chemical exposure.

4. The method of any one of aspects 1-3, wherein the ocular injury or ocular surgery causes a corneal wound or a conjunctival wound.

5. The method of any one of aspects 1-4, wherein the ocular injury increases risk of corneal or conjunctival fibrosis or scarring.

6. The method of any one of aspects 1-5, wherein multiple cycles of treatment are administered to the subject.

7. The method of aspect 6, wherein the verteporfin is administered to the subject intermittently.

8. The method of aspect 7, wherein the verteporfin is administered no more frequently than biweekly.

9. The method of any one of aspects 6-8, wherein the verteporfin is administered for at least one month, at least two months, or at least three months.

10. The method of any one of aspects 1-9, wherein said administering comprises administering the verteporfin topically or by subconjunctival injection.

11. The method of aspect 10, wherein each administration of verteporfin provides a dose ranging from 0.001 mg to 10 mg of verteporfin.

12. The method of aspect 11, wherein each administration of verteporfin provides a dose ranging from 0.01 mg to 1 mg of verteporfin.

13. The method of aspect 11 or 12, wherein the verteporfin is in a volume ranging from 0.1 ml to 1 ml.

14. The method of any one of aspects 10-13, wherein the verteporfin is administered topically on the cornea or locally under the conjunctiva.

15. The method of any one of aspects 1-14, wherein the verteporfin is administered before, during, or after an ocular surgical procedure that increases the risk of developing fibrosis in ocular tissues.

16. The method of aspect 15, wherein the ocular surgical procedure comprises incision of the conjunctiva.

17. The method of aspect 15 or 16, wherein the ocular surgical procedure is glaucoma filtration surgery (trabeculectomy), minimally invasive glaucoma surgery (MIGS), insertion of a glaucoma drainage implant, insertion of a Xen gel stent, or insertion of other stents or devices in the subconjunctival space, a revision of a trabeculectomy, a revision of MIGS, a revision of a glaucoma drainage implant, a revision of a Xen gel stent, or revision of other stents or devices in the subconjunctival space

18. The method of aspect 15, wherein the ocular surgical procedure comprises incisional surgery or bleb needling to revise a previous trabeculectomy, previous tube shunt surgery, or other previous incisional surgery.

19. The method of any one of aspects 1-18, wherein a first dose of verteporfin is administered before, during, or after the ocular surgical procedure; and subsequent doses of verteporfin are administered no more frequently than biweekly up to postoperative month three.

20. The method of aspect 19, wherein up to five subsequent doses of verteporfin are administered.

21. The method of aspect 19 or 20, further comprising administering additional doses of verteporfin after postoperative month three.

22. The method of aspect 20 or 21, further comprising administering one, two, three, four, five, six, seven, eight, or nine additional doses of verteporfin after the fifth dose of verteporfin.

23. The method of aspect 22, wherein the additional doses of verteporfin are administered monthly.

24. The method of any one of aspects 1-23, wherein administration of the verteporfin is performed in conjunction with bleb needling or other manipulation of the conjunctiva, performed in the office or in the operating room.

25. The method of any one of aspects 1-24, wherein the verteporfin is administered after an ocular injury that increases the risk of developing fibrosis or scarring of ocular tissues.

26. A method of treating fibrosis in ocular tissues, the method comprising: identifying a subject who has fibrosis in ocular tissues or is at risk of developing fibrosis or scarring in ocular tissues after a previous ocular surgery or ocular injury;
administering to the subject a first dose of verteporfin; and
administering to the subject subsequent doses of verteporfin no more frequently than biweekly, wherein up to five doses of verteporfin are administered to the subject.

27. The method of aspect 26, wherein each administration of verteporfin provides a dose ranging from 0.001 mg to 10 mg of verteporfin.

28. The method of aspect 27, wherein each administration of verteporfin provides a dose ranging from 0.01 mg to 1 mg of verteporfin.

29. The method of aspect 27 or 28, wherein each administration of verteporfin provides a dose ranging from 0.01 mg to 1 mg of verteporfin in a volume ranging from 0.1 ml to 1 ml.

30. The method of any one of aspects 26-29, wherein verteporfin is administered topically on the cornea or under the conjunctiva.

31. The method of any one of aspects 26-30, comprising administering one, two, three, four, five, six, seven, eight, or nine additional doses of verteporfin after the fifth dose of verteporfin.

32. The method of aspect 31, wherein the additional doses of verteporfin are administered monthly.

33. The method of any one of aspects 26-32, wherein each administration of verteporfin is performed in conjunction with bleb needling or other manipulation of the conjunctiva, performed in an office or in an operating room.

34. The method of any one of aspects 26-33, wherein the ocular surgical procedure is glaucoma filtration surgery (trabeculectomy), minimally invasive glaucoma surgery (MIGS), insertion of a glaucoma drainage implant, insertion of a Xen gel stent, or insertion of other stents or devices in the subconjunctival space, a revision of a trabeculectomy, a revision of MIGS, a revision of a glaucoma drainage implant, a revision of a Xen gel stent, revision of other stents or devices in the subconjunctival space, or other conditions where the conjunctiva is incised.

35. The method of any one of aspects 26-34, wherein the ocular injury is caused by physical trauma or a chemical exposure.

36. A method of reducing corneal scarring in an eye of a subject after a corneal injury, the method comprising administering a therapeutically effective amount of verteporfin to the subject.

37. A composition comprising verteporfin for use in a method of treating fibrosis or scarring in ocular tissues or reducing risk of developing fibrosis or scarring in ocular tissues.

38. A composition comprising verteporfin for use in a method of treating fibrosis or scarring in ocular tissues, the method comprising:
identifying a subject who has fibrosis in ocular tissues or is at risk of developing fibrosis or scarring in ocular tissues after a previous ocular surgery or ocular injury;
administering to the subject a first dose of verteporfin; and
administering to the subject subsequent doses of verteporfin no more frequently than biweekly, wherein up to five doses of verteporfin are administered.

39. A composition comprising verteporfin for use in a method of treating a subject after a corneal injury to reduce corneal scarring.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### Verteporfin for Glaucoma Surgery

Verteporfin, a benzoporphyrin derivative, has been shown to prevent fibrosis in several human organs, including the lung. Recently, a study showed that injection of verteporfin into skin wounds in mice prevented scar formation and instead promoted wound regeneration with restoration of normal glands, extracellular matrix, and mechanical strength (Mascharak et al. (2021) Science 372: eaba2374). Verteporfin was approved in 2000 as a photosensitizer for photodynamic therapy to treat abnormal blood vessels in the eye caused by the wet form of macular degeneration. The mechanism of action for the FDA approved drug indication is not related to the mechanism of action by which verteporfin prevents scarring.

Here we describe the use of verteporfin, e.g., single or repeated dosing, for treating or reducing conjunctival fibrosis after glaucoma surgery, e.g., trabeculectomy. Currently, the most commonly used agents, 5-fluorouracil and mitomycin C (MMC), are antimetabolites which can cause cytotoxic side effects and causes complications such as wound leak, hypotony and endophthalmitis. Verteporfin has already been FDA approved as an intravenous injection with photodynamic therapy to treat choroidal neovascularization in the eye and has been shown to be safe for that indication. It can be dosed safely in the skin with minimal side effects, as shown in previous animal studies (Mascharak et al., supra).

### Example 2

Verteporfin Reduces Expression of α-Smooth Muscle Actin in hMSCs

Human mesenchymal stem cells (hMSCs) were plated in culture. The next day, the following treatments were added in duplicate: 1 μg/mL Verteporfin, 10 μg/mL Verteporfin, 10 ng/ml transforming growth factor beta-1 (TGFB-1), 1 μg/mL Verteporfin+10 ng/ml TGFB-1, 10 μg/mL Verteporfin+10 ng/ml TGFB-1, Dimethyl sulfoxide (DMSO), and cells without treatment. After 24 hours, the medium was removed, the wells were washed with PBS and then the RNA of the cells were extracted using Trizol and a Quiagen kit. Quantitative polymerase chain reaction (qPCR) was performed.

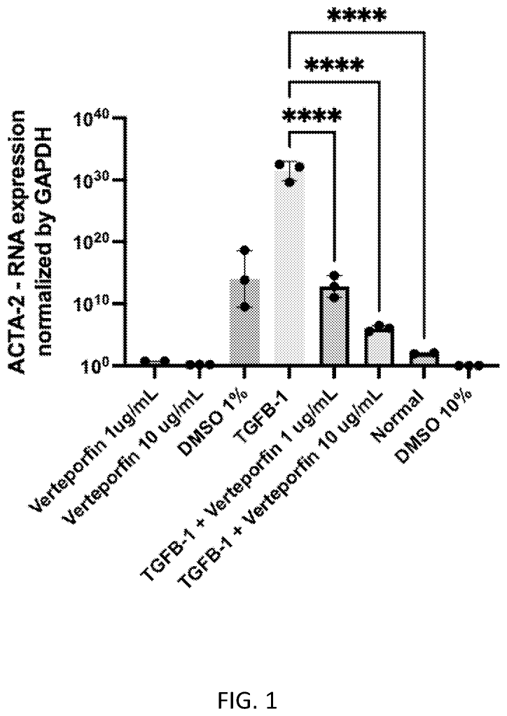

As shown in FIG. 1, TGFB-1 activation leads to the differentiation of mesenchymal stem cells into myofibroblasts, inducing expression of α-smooth muscle actin (α-SMA) and the extracellular matrix proteins such as collagen which may lead to scarring. The addition of TGFB-1 statistically increased the expression of alpha-SMA (ACTA-2) after 24 hours. Treating cells with TGFB-1 and Verteporfin statistically reduced the expression of alpha-SMA in a dose dependent manner, suggesting that verteporfin may reduce scarring and fibrosis.

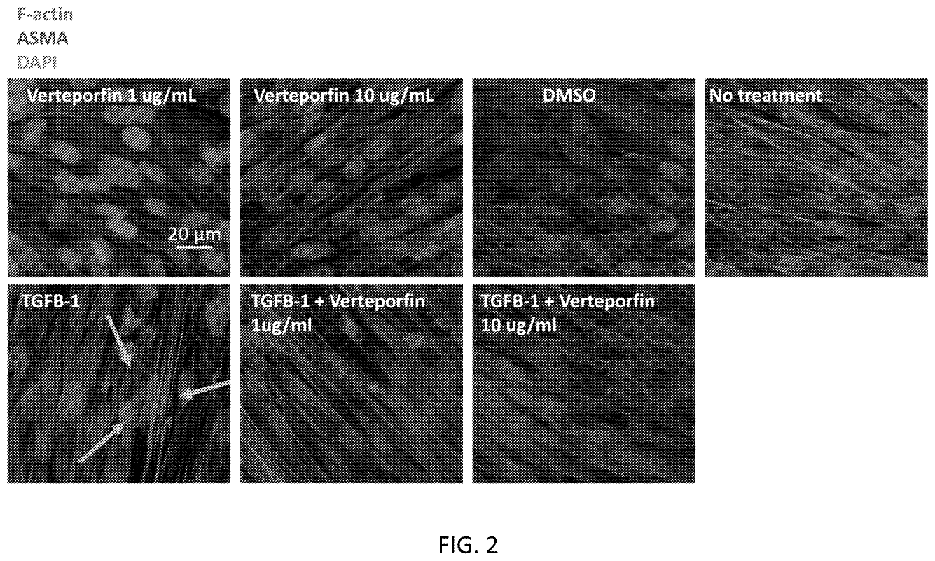

The hMSCs, treated with 10 ng/ml TGFB-1 with or without verteporfin and DMSO for 24 hours, were fixed and stained for alpha smooth muscle actin (ASMA) the next day. As shown in FIG. 2, Verteporfin reduced the expression of ASMA, suggesting that it can reduce scarring and the fibrotic response.

### Example 3

Verteporfin does not Increase Expression of Ki67 in hMSCs

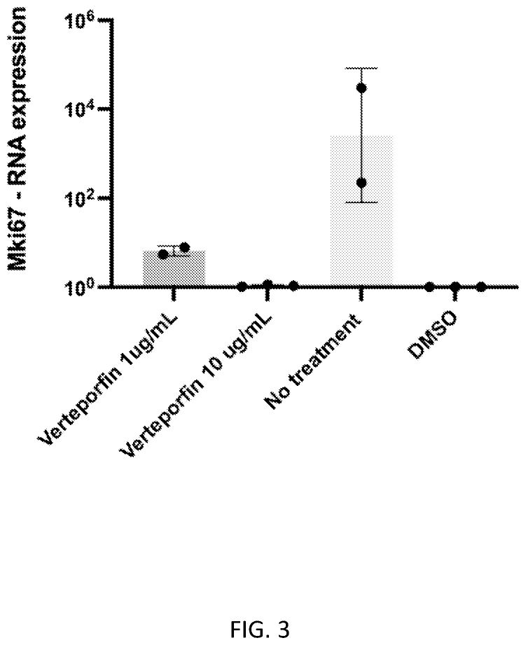

The hMSCs were cultured on plates. The next day, the treatments were added in duplicate: Verteporfin 1 μg/mL, Verteporfin 10 μg/mL, DMSO, and cells without treatment. After 24 hours, the medium was removed, the wells were washed with PBS and then the RNA of the cells were extracted using Trizol and Quiagen kit. qPCR was performed. As shown in FIG. 3, the addition of Verteporfin did not increase the expression of ki67 (Mki67) a marker of cell proliferation.

### Example 4

Verteporfin Is Not Cytotoxic to hMSCs

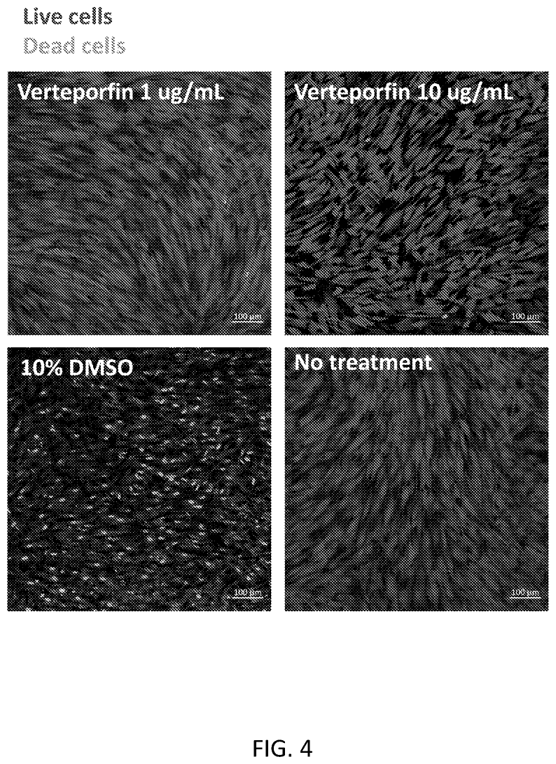

The hMSCs were treated with verteporfin at concentrations of 1 μg/mL and 10 μg/mL for 24 hours. Cells were treated with 10% DMSO for 40 minutes. The next day, the live/dead solution was added to the cells. As shown in FIG. 4, verteporfin at 1 μg/mL and 10 μg/mL is not cytotoxic to hMSCs.

### Example 5

### Efficacy of Verteporfin in Reducing Scarring and Increasing Bleb Survival

The efficacy of verteporfin in reducing scarring and increasing bleb survival was evaluated in a rabbit model of glaucoma filtration surgery. Glaucoma filtration surgery was performed in both eyes of New Zealand White rabbits. In one eye, various concentrations of verteporfin or Mitomycin C was injected subconjunctivally into the bleb after surgery. In the contralateral eye, BSS was injected after surgery as a control. Bleb appearance and survival were evaluated postoperatively at least twice a week. Bleb survival was the primary endpoint.

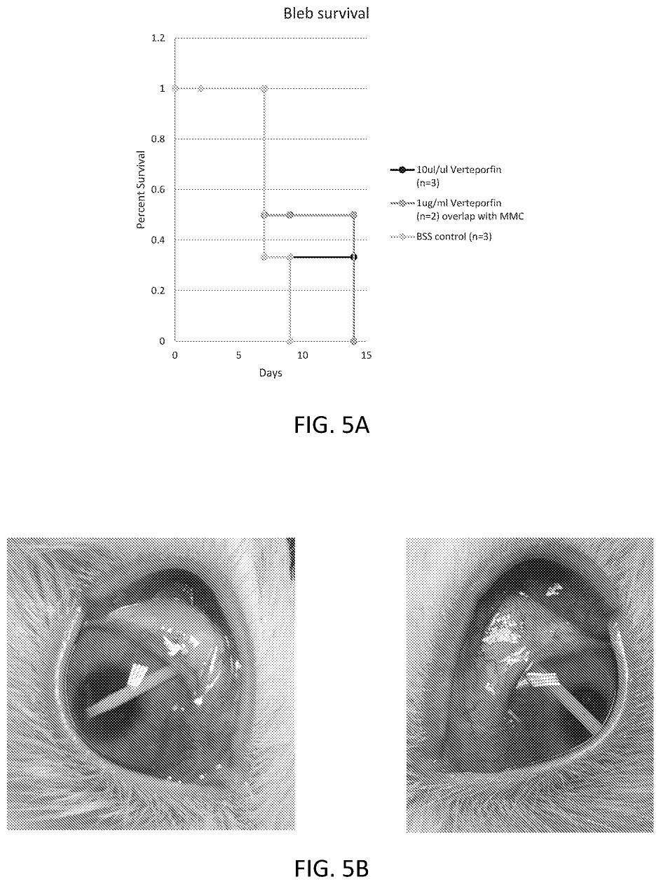

As shown in FIG. 5A, the results show a trend towards increased survival in the verteporfin treated groups over the control. There were no post-surgical complications in all groups, including corneal edema, bleb leak, anterior chamber inflammation, iris involvement, cataract or infection (FIG. 5B).

### Example 6

Efficacy of Verteporfin in Reducing Corneal Scarring after Corneal Injury

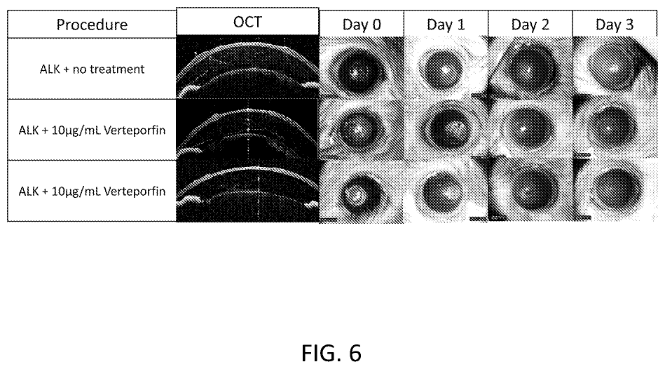

Anterior lamellar keratectomy (ALK) with a cut depth of around 50% was performed in a rat model of corneal injury. 10 μg/mL verteporfin (in Provisc gel) was subconjunctivally injected after ALK on Day 0. Eyes treated with verteporfin showed reduced corneal scarring at day 3 compared with eyes that were not treated (FIG. 6).

What is claimed is:

1. A method of treating fibrosis or reducing risk of developing fibrosis or scarring in an eye of a subject after an ocular injury or ocular surgery, the method comprising administering a therapeutically effective amount of verteporfin to the subject.

2. The method of claim 1, wherein the ocular surgery is glaucoma surgery.

3. The method of claim 1, wherein the ocular injury is caused by physical trauma or a chemical exposure.

4. The method of any one of claims 1-3, wherein the ocular injury or ocular surgery causes a corneal wound or a conjunctival wound.

5. The method of any one of claims 1-4, wherein the ocular injury increases risk of corneal or conjunctival fibrosis or scarring.

6. The method of any one of claims 1-5, wherein multiple cycles of treatment are administered to the subject.

7. The method of claim 6, wherein the verteporfin is administered to the subject intermittently.

8. The method of claim 7, wherein the verteporfin is administered no more frequently than biweekly.

9. The method of any one of claims 6-8, wherein the verteporfin is administered for at least one month, at least two months, or at least three months.

10. The method of any one of claims 1-9, wherein said administering comprises administering the verteporfin topically or by subconjunctival injection.

11. The method of claim 10, wherein each administration of verteporfin provides a dose ranging from 0.001 mg to 10 mg of verteporfin.

12. The method of claim 11, wherein each administration of verteporfin provides a dose ranging from 0.01 mg to 1 mg of verteporfin.

13. The method of claim 11 or 12, wherein the verteporfin is in a volume ranging from 0.1 ml to 1 ml.

14. The method of any one of claims 10-13, wherein the verteporfin is administered topically on the cornea or locally under the conjunctiva.

15. The method of any one of claims 1-14, wherein the verteporfin is administered before, during, or after an ocular surgical procedure that increases the risk of developing fibrosis in ocular tissues.

16. The method of claim 15, wherein the ocular surgical procedure comprises incision of the conjunctiva.

17. The method of claim 15 or 16, wherein the ocular surgical procedure is glaucoma filtration surgery (trabeculectomy), minimally invasive glaucoma surgery (MIGS), insertion of a glaucoma drainage implant, insertion of a Xen gel stent, or insertion of other stents or devices in the subconjunctival space, a revision of a trabeculectomy, a revision of MIGS, a revision of a glaucoma drainage implant, a revision of a Xen gel stent, or revision of other stents or devices in the subconjunctival space

18. The method of claim 15, wherein the ocular surgical procedure comprises incisional surgery or bleb needling to revise a previous trabeculectomy, previous tube shunt surgery, or other previous incisional surgery.

19. The method of any one of claims 1-18, wherein a first dose of verteporfin is administered before, during, or after the ocular surgical procedure; and subsequent doses of verteporfin are administered no more frequently than biweekly up to postoperative month three.

20. The method of claim 19, wherein up to five subsequent doses of verteporfin are administered.

21. The method of claim 19 or 20, further comprising administering additional doses of verteporfin after postoperative month three.

22. The method of claim 20 or 21, further comprising administering one, two, three, four, five, six, seven, eight, or nine additional doses of verteporfin after the fifth dose of verteporfin.

23. The method of claim 22, wherein the additional doses of verteporfin are administered monthly.

24. The method of any one of claims 1-23, wherein administration of the verteporfin is performed in conjunction with bleb needling or other manipulation of the conjunctiva, performed in the office or in the operating room.

25. The method of any one of claims 1-24, wherein the verteporfin is administered after an ocular injury that increases the risk of developing fibrosis or scarring of ocular tissues.

26. A method of treating fibrosis in ocular tissues, the method comprising: identifying a subject who has fibrosis in ocular tissues or is at risk of developing fibrosis or scarring in ocular tissues after a previous ocular surgery or ocular injury;
administering to the subject a first dose of verteporfin; and
administering to the subject subsequent doses of verteporfin no more frequently than biweekly, wherein up to five doses of verteporfin are administered to the subject.

27. The method of claim 26, wherein each administration of verteporfin provides a dose ranging from 0.001 mg to 10 mg of verteporfin.

28. The method of claim 27, wherein each administration of verteporfin provides a dose ranging from 0.01 mg to 1 mg of verteporfin.

29. The method of claim 27 or 28, wherein each administration of verteporfin provides a dose ranging from 0.01 mg to 1 mg of verteporfin in a volume ranging from 0.1 ml to 1 ml.

30. The method of any one of claims 26-29, wherein verteporfin is administered topically on the cornea or under the conjunctiva.

31. The method of any one of claims 26-30, comprising administering one, two, three, four, five, six, seven, eight, or nine additional doses of verteporfin after the fifth dose of verteporfin.

32. The method of claim 31, wherein the additional doses of verteporfin are administered monthly.

33. The method of any one of claims 26-32, wherein each administration of verteporfin is performed in conjunction with bleb needling or other manipulation of the conjunctiva, performed in an office or in an operating room.

34. The method of any one of claims 26-33, wherein the ocular surgical procedure is glaucoma filtration surgery (trabeculectomy), minimally invasive glaucoma surgery (MIGS), insertion of a glaucoma drainage implant, insertion of a Xen gel stent, or insertion of other stents or devices in the subconjunctival space, a revision of a trabeculectomy, a revision of MIGS, a revision of a glaucoma drainage implant, a revision of a Xen gel stent, revision of other stents or devices in the subconjunctival space, or other conditions where the conjunctiva is incised.

35. The method of any one of claims 26-34, wherein the ocular injury is caused by physical trauma or a chemical exposure.

36. A method of reducing corneal scarring in an eye of a subject after a corneal injury, the method comprising administering a therapeutically effective amount of verteporfin to the subject.

37. A composition comprising verteporfin for use in a method of treating fibrosis or scarring in ocular tissues or reducing risk of developing fibrosis or scarring in ocular tissues.

38. A composition comprising verteporfin for use in a method of treating fibrosis or scarring in ocular tissues, the method comprising:
identifying a subject who has fibrosis in ocular tissues or is at risk of developing fibrosis or scarring in ocular tissues after a previous ocular surgery or ocular injury;
administering to the subject a first dose of verteporfin; and
administering to the subject subsequent doses of verteporfin no more frequently than biweekly, wherein up to five doses of verteporfin are administered.

39. A composition comprising verteporfin for use in a method of treating a subject after a corneal injury to reduce corneal scarring.
